(19) United States
(12) Patent Application Publication
(10) Pub. No.: US 2023/0398111 A1
(43) Pub. Date: Dec. 14, 2023

(54) ORAL FORMULATIONS OF NINTEDANIB AND METHOD OF MANUFACTURING THEREOF

(71) Applicant: BDR PHARMACEUTICALS INTERNATIONAL PRIVATE LIMITED, Mumbai (IN)

(72) Inventors: Shah Dharmesh MAHENDRABHAI, Mumbai (IN); Badiger Aravind Manappa, Halol (IN); Sharma Mukeshkumar SUBHASHCHANDRA, Halol (IN); Trivedi Madhavkumar DILIPBHAI, Halol (IN); Choksi Rakshit KETANBHAI, Halol, IN (US); Panchal Samirkumar BABULAL, Halol (IN); Vora Pratik ASHWINBHAI, Halol (IN); Patel Harikrishnabhai VINUBHAI, Halol (IN); Shah Shreyash PRAKASHKUMAR, Halol (IN); Gandhi Prashant KANAIYALAL, Halol (IN)

(21) Appl. No.: 18/249,344

(22) PCT Filed: Oct. 14, 2021
(86) PCT No.: PCT/IN2021/050984

§ 371 Date: Apr. 17, 2023

(30) Foreign Application Priority Data
Oct. 15, 2020 (IN) 202021044873

(51) Int. Cl. A61K 31/496; A61K 9/48

(52) U.S. Cl. CPC A61K 31/496; A61K 9/4858; A61K 9/4825; A61K 9/4875

(57) ABSTRACT

The present invention relates to an oral soft-gelatin capsule dosage form of Nintedanib and pharmaceutically acceptable salt thereof. The invention also relates to provide patient-compliant, economical and technically advanced dosage form over existing marketed dosage form as well as nearest prior-arts. Moreover, the dissolution rate and stability of the patient compliant Nintedanib formulation, prepared as per the present invention, is proven higher when compared to prior art inventions. Furthermore, the present invention also provides an oily dispersion without using lecithin is prepared by a process which is relatively simple, easy to commercially manufacture, and functionally reproducible.

### FIELD OF THE INVENTION

The present invention relates to oral capsule formulation of Nintedanib and pharmaceutically acceptable salt thereof. This present invention discloses soft-gelatin capsule as well as hard-gelatin capsule formulation of Nintedanib and pharmaceutically acceptable salt thereof. Further, the present invention relates to providing an economical and technically advanced dosage form over existing dosage forms.

### BACKGROUND OF THE INVENTION

Nintedanib (CAS: 656247-17-5) is a member of the class of oxindoles that is a small molecule kinase inhibitor used (in the form of its “ethylsulfonate salt” also pronounced as “esyalte salt”) for the treatment of idiopathic pulmonary fibrosis (IPF), chronic fibrosing interstitial lung diseases (ILDs) with a progressive phenotype and slowing the rate of decline in pulmonary function in patients with systemic sclerosis-associated interstitial lung disease (SSc-ILD). Nintedanib esylate (CAS: 656247-18-6) is a bright yellow powder with an empirical formula of C₃₁H₃₃N₅O₄·C₂H₆O₃S and a molecular weight of 649.76 g per mol. Nintedanib esylate is chemically known as methyl-2-hydroxy-3-[N-[4-[methyl-[2-(4-methylpiperazin-1-yl)acetyl]amino]phenyl]-C-phenylcarbonimidoyl]-1H-indole-6-carboxylate ethanesulfonic acid and is represented structurally as below:

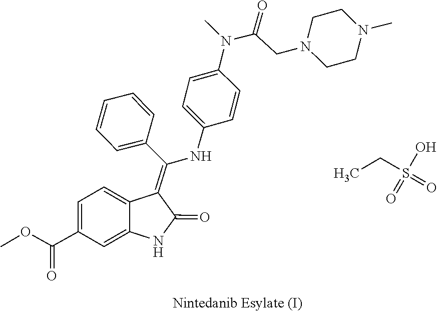

Nintedanib was first disclosed in U.S. Pat. No. 6,762,180 which is a patent assigned to Boehringer Ingelheim. This patent disclosed Nintedanib as well as process for preparation thereof wherein the reaction of methyl (Z)-1-acetyl-3-(ethoxyphenylmethylene)-oxindole-6-carboxylate with N-(4-aminophenyl)-N,4-dimethyl-1-piperazine acetamide in dimethylformamide is taken place, that is followed by the treatment with piperidine to obtain Nintedanib free base.

Nintedanib esylate was disclosed later on in U.S. Pat. No. 7,119,093 which was filed during year 2003 and claiming the salt of Nintedanib as 3-Z-[1-(4-(4-(N-((4-methyl-piperazin-1-yl)-methylcarbonyl)-N-methyl-amino)-anilino)-1-phenyl-methylene]-6-methoxycarbonyl-2-indolinone monoethanesulphonate, preferably in crystalline form, characterized by a melting point of T_{m.p.}=305±5° C. (determined by DSC; evaluation by peak maximum; heating rate: 10° C./min).

Nintedanib esylate is the presently marketed salt in the formulation having brand name OFEV® which is developed by Boehringer Ingelheim, available in multiple strengths for oral administration capsule, which was first approved by the USFDA on Oct. 15, 2014. In the US, primarily, it was approved by the USFDA for the treatment of the treatment of idiopathic pulmonary fibrosis (IPF) and to slow declining pulmonary function in patients with systemic sclerosis-associated interstitial lung disease. Nintedanib was granted orphan drug designation in the US by the FDA for the treatment of idiopathic pulmonary fibrosis in June 2011 until 15 Oct. 2021. In March 2020, it was approved for use in the United States to treat chronic fibrosing (scarring) interstitial lung diseases (ILD) with a progressive phenotype (trait). It is the first treatment, for this group of fibrosing lung diseases that worsen over time. In the EU, Nintedanib is indicated in combination with docetaxel for the treatment of adult patients with metastatic, locally advanced, or locally recurrent non-small cell lung cancer of adenocarcinoma histology who have already tried first-line therapy.

OFEV® is available in two different strengths of 100 mg and 150 mg. The recommended dosage of Nintedanib esylate is 150 mg twice daily approximately 12 hours apart taken with food. However, the recommended dosage may be varied in patients with mild hepatic impairment (Child Pugh A) as 100 mg twice daily approximately 12 hours apart taken with food. In case of management of adverse reactions, temporary dose reduction to 100 mg or discontinuation of the composition is employed. Therefore, to reduce such incidences prior to treatment initiation, liver function tests are conducted in all patients and a pregnancy test in females of reproductive potential.

### Effect of Lecithin as an Essential Excipient is Reported in U.S. Pat. No. 9,907,756 (WO2009/147212)

Above composition was first disclosed by Boehringer Ingelheim in U.S. Pat. No. 9,907,756 (WO2009/147212). As per this patent, the said marketed composition is a lipid suspension of Nintedanib esylate in 1 to 90 wt. % of medium chain triglycerides, 1 to 30 wt. % of hard fat and 0.1 to 10 wt. % of lecithin. Further, this patent has also carried out study of effect of lecithin wherein it was checked on the in-vitro dissolution behavior (in % of dissolution) over time (in minutes) of soft gelatin capsules and following results were obtained. Above results shows a clear-cut indication that absence of lecithin in soft gelatin capsule formulation yields poorest dissolution rate when compared to the highest amount of lecithin. Hence, lecithin is an essential part of this invention as it is used as glidant/solubilizing agent.

From the literatures, it is known that Nintedanib has poor solubility and stability at neutral conditions, hence it cannot be formulated as a solution. Nintedanib and its esylate salt has poor solubility at neutral conditions which in turn is responsible for lower bioavailability of only 4-7%, which greatly limits its clinical use. To overcome the problem of solubility and stability, several approaches are reported after above-mentioned innovator composition.

CN108078952 discloses a soft capsule comprising suspension of Nintedanib esylate having the size distribution range of the D₉₀ from 40 μm to 80 μm. The reduction of particle size of active, its incorporation in the formulation, and the maintenance of the size needs special process of manufacture and is costly. Also, the dissolution profile depends on particle size of active ingredient.

WO2019/106692 filed by Sun Pharmaceuticals discloses an oral pharmaceutical suspension adapted for encapsulation in soft gelatin capsules consists of medium chain triglycerides, a surfactant having hydrophilic-lipophilic balance value ranging from 8 to 10 and lecithin. Similar to earlier prior-arts, lecithin is again an essential part of this invention.

CN107184549 provides a capsule containing Nintedanib self micro-emulsion wherein the droplet size of the microemulsion is between 10 and 100 nm and the carrier medium is designed to spontaneously form an emulsion in the stomach thereby facilitating absorption of Nintedanib. These systems are self micro-emulsifying drug delivery system (SMEDDS). A major drawback of such system is that they have to be accurately prepared and even slight variation in composition will not lead to formation of emulsion in the stomach and thus destroying tits beneficial properties. Also, controlling the particle size of active plays a major role in the system. Hence, this composition may be proven as costlier.

WO2019/197961 filed by Intas Pharmaceuticals discloses a soft-gelatin capsule comprises medium chain triglycerides, either Lauroyl polyoxyl-6 glyceride or Hydrogenated vegetable oil along with Lecithin. It is surprising to note this patent application has claimed the same both excipients which were already disclosed and anticipated in U.S. Pat. No. 9,907,756 with soft gelatin capsule formulation of Nintedanib esylate. Similar to earlier prior-arts, lecithin is again an essential part of this invention. Hence, WO2019/197961 may not be considered as an invention due to lack of novelty and inventiveness.

CN105963268 discloses a dispersible tablet comprising Nintedanib and other pharmaceutically acceptable excipients necessary for dispersion of tablet. The drugs having low solubility may not dissolve in the excipients of the conventional dosage form and thus leading to no content uniformity and very high deviation.

WO2020/079706 filed by Cipla Limited claims a soft-gelatin capsule formulation of Nintedanib which comprises medium chain triglycerides, lecithin and carrier system. The carrier system consists of triglycerides of saturated fatty acids containing 8 to 10 carbon atoms. Example of above includes hard fat. As per the last claim of this patent application, the inactive ingredients of the composition comprise 30% to 50% medium chain triglycerides; 10% to 30% of hard fat and 11% of lecithin. Hence, similar to earlier prior-arts, lecithin is again an essential part of this invention.

Although the prior arts disclose different types of compositions which address the solubility issues of Nintedanib. It was important to note that Lecithin is an important excipient which was essentially deployed in almost all prior-inventions. In addition, many of these solubility enhancing techniques involve critical and tedious manufacturing process, make use of many excipients or are modified according to the specific physicochemical characteristics of Nintedanib.

Hence, the inventors of the present invention felt that still there is an unmet need to develop a dosage form of Nintedanib, which overcome the drawbacks of prior available formulations, has good physical stability, is cost effective, can be produced by simple manufacturing techniques and can maximize the solubility of Nintedanib from such dosage form and thereby improve its bioavailability.

### OBJECTIVES OF THE INVENTION

The primary object of the present invention is to provide improved oral dosage formulations of Nintedanib and pharmaceutically acceptable salt thereof in soft-gelatin capsule.

Another object of the present invention is to provide economical and advanced dosage forms over existing dosage form and prior-arts.

Yet another object of the present invention is to provide formulation of Nintedanib and pharmaceutically acceptable salt thereof, which is devoid of Lecithin. Lecithin is an essential inactive ingredient in any of the reported prior-arts of Nintedanib soft-gelatin capsule. Increase in amount of Lecithin yields faster dissolution rate as per prior-art publications.

One more object of the present invention is to provide a process for preparation of oral dosage formulation of Nintedanib and pharmaceutically acceptable salt thereof as soft-gelatin capsule.

Yet another object of the present invention is to provide Nintedanib and pharmaceutically acceptable salt thereof oral dosage form with enhanced faster dissolution profile.

One more object of the present invention is to provide an oral pharmaceutical formulation comprising of oily dispersion showing 90% of drug dissolution within 15 minutes and total impurities less than 0.2% after stability study of 9 months at (40±2)° C. and (75±5)% RH.

### SUMMARY OF THE INVENTION

Despite of extensive research on Nintedanib as reported in prior-art publications, there is an unmet need to develop a patient compliant oral soft-gelatin capsule composition for Nintedanib with technical advancement which provide fast dissolution for immediate action as well as good stability for long-term storage.

Accordingly, the present invention provides an oral composition of Nintedanib preferably as homogenous dispersion dosage form with pharmaceutically acceptable excipients and method of preparation thereof.

The prime objective of the present invention is that the pharmaceutical composition establishes a very fast release during initial dissolution owing to kind of suspending or dispersing agents as well as vehicle used in the formulation and thereby producing an immediate burst release which in turn immediate absorption of the drug into the blood stream. This action will help to increase bioavailability of Nintedanib and pharmaceutically acceptable salt thereof in very short time.

In one general embodiment of the present invention, a pharmaceutical composition as per the present invention is in the form of capsule formulation. Preferably, this capsule formulation is soft-gelatin capsule formulation and optionally, it is hard-gelatin or vegetable source containing hard capsule.

In yet another embodiment of the present invention, a pharmaceutical composition as per the present invention comprises Nintedanib and one or more pharmaceutically acceptable excipients wherein the composition is in the form of a homogenous dispersion.

In another general embodiment of the present invention, a pharmaceutical composition as per the present invention comprises Nintedanib or pharmaceutically acceptable salt thereof. Preferably, Nintedanib salt used in Nintedanib esylate as active ingredient.

In one embodiment of the present invention, wherein the active ingredient incorporated in the pharmaceutical composition is in crystalline form. Optionally, active ingredient is present in amorphous form.

In another embodiment of the present invention, wherein suspending or dispersing agent helps to produce homogenous dispersion.

In yet another embodiment of the present invention, a pharmaceutical composition as per the present invention comprises Nintedanib and one or more pharmaceutically acceptable excipients wherein the composition is in the form of an oily homogenous dispersion.

In another embodiment of the present invention, a pharmaceutical composition as per the present invention is devoid of Lecithin which is repeatedly used in prior-art publications as solubilizer to enhance the dissolution rate. Also, it may be important to note that Lecithin derived from the source of Soya like Soya-Lecithin may provide an immune response to the patients who are allergic to the same. Hence, removing of Lecithin provides patient compliance as well.

Embodiments of the pharmaceutical composition may include Nintedanib as an active ingredient with one or more selected from pharmaceutically acceptable excipients like diluent, vehicles, stabilizers/anti-oxidants, suspending or dispersing agents, permeability enhancers, preservatives, glidants, active carriers, anti-caking agents, wetting agents, preservatives, buffering agents and the like.

In another embodiment of the present invention, pharmaceutical composition is in the form of an oral dispersion comprising:
a) Melting Labrafil M 2130 CS;
b) mixing Labrafac Lipofile WL 1349 along with Nintedanib esylate with melted Labrafil M 2130 CS obtained in step-1 and stirred continuously until it became homogeneous dispersion;
c) The homogenous dispersion prepared in step-2, was then encapsulated in soft-gelatin capsule.

The details of one or more embodiments of the invention are set forth in the description below. Other features of the invention will be apparent from the description.

### BRIEF DESCRIPTION OF THE DRAWINGS

FIG. 1: shows a graphical representation of comparative dissolution profile (as reported in test example-3) of the present invention against the nearest prior-arts.

### DETAILED DESCRIPTION OF THE INVENTION

The following detailed description of the present subject matter the various embodiments. These embodiments are described in sufficient detail to enable those skilled in the art to practice the present subject matter. Other embodiments may be utilized and structural, logical, and electrical changes may be made without departing from the scope of the present subject matter.

References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Nintedanib, chemically known as, methyl-2-hydroxy-3-[N-[4-[methyl-[2-(4-methylpiperazin-1-yl)acetyl]amino]phenyl]-C-phenylcarbonimidoyl]-1H-indole-6-carboxylate, is a small molecule kinase inhibitor having molecular weight of 649.76 g per mol. Nintedanib is considered a Bio-pharmaceutics Classification System (BCS) Class II or IV drug substance due to low aqueous solubility at neutral pH. The mean Nintedanib is a drug with very much low solubility.

In accordance with the present invention, a pharmaceutical composition of Nintedanib comprising of Nintedanib as an active ingredient with pharmaceutically acceptable excipients. Preferable salt used in the present invention is Nintedanib esylate.

In accordance with the present invention, the active ingredient incorporated in the pharmaceutical composition is in crystalline form. Optionally, active ingredient is present in amorphous form.

The term “pharmaceutically acceptable excipients” as used herein, refers to excipients those are routinely used in pharmaceutical compositions. The pharmaceutically acceptable excipients may comprise of diluent, vehicles/solubility enhancing agents, stabilizers/anti-oxidants, suspending or dispersing agents, permeability enhancers, preservatives, glidants, active carriers, anti-caking agents, wetting agents, preservatives, buffering agents and combinations thereof.

At the time of preparation of the dosage form, the excipients of oral dispersion are mainly divided into following parts according to their uses in the manufacturing process (A) drug part, (B) vehicles or solubilizers and (C) suspending or dispersing agents.

Suitable vehicles may include one or more from Soybean Oil, Olive Oil, Sesame Oil, medium chain fatty acid triglycerides like Labrafac® Lipophile WL 1349, Labrafac® CC, Geloil™, Peceol®, Lauroglycol™ 90, Lauroglycol™ FCC, Capryol™ 90, Capryol™ PGMC, Plurol® Oleique CC497, Labrasol®, Lauroyl macrogol (or polyoxyl) 6 glycerides like Labrasol® ALF and the like.

Suitable suspending or dispersing agents may include one or more from Lauroyl macrogol (or polyoxyl) 6 glycerides like Labrafil® M 1944 CS, Labrafil® M 2125 CS, Labrafil® M 2130 CS, Gelucire® 44/14, Lauroglycol™ 90, Lauroglycol™ FCC, Maisine® CC, Peceol™, Transcutol® HP and the like.

In accordance with the present invention, a pharmaceutical composition as per the present invention comprises Nintedanib and one or more pharmaceutically acceptable excipients wherein the composition is in the form of a homogenous oily dispersion.

A pharmaceutical composition as per the present invention is devoid of Lecithin which is repeatedly used excipient in prior-art publications as solubilizer to enhance the dissolution rate.

Suitable stabilizers or anti-oxidants may include one or more from citric acid, butylated hydroxytoluene, butylated hydroxy anisole, sodium bisulphite, ascorbic acid, L-cysteine, magnesium bisulfite, sodium metabisulfite, tocopherol, ubiquinol, β-carotenes, uric acid, lipoic acid, propyl gallate, thiourea, glutathione and the like.

Suitable preservative may include one or more from propylene glycol, disodium EDTA, benzalkonium chloride, benzoic acid, butyl paraben, methyl paraben, propyl paraben, sodium benzoate and the like.

Suitable anti-caking agents may include one or more from colloidal silicon dioxide, tribasic calcium phosphate, magnesium trisilicate, starch and the like.

Suitable wetting agents may include one or more from the group comprising anionic, cationic, nonionic, or zwitterionic surfactants, or combinations thereof. Suitable examples of wetting agents are sodium lauryl sulphate, cetrimide, polyethylene glycols, polyoxyethylene-polyoxypropylene block copolymers such as poloxamers, polyglycerin fatty acid esters such as decaglyceryl monolaurate and decaglyceryl monomyristate, sorbitan fatty acid esters such as sorbitan monostearate, polyoxyethylene sorbitan fatty acid esters such as polyoxyethylene sorbitan monooleate, polyethylene glycol fatty acid esters such as polyoxyethylene monostearate, polyoxyethylene alkyl ethers such as polyoxyethylene lauryl ether, polyoxyethylene castor oil and the like.

Suitable permeability enhancers may include one or more from the group comprising alcohols, Polyols, short chain glycerides, amines, amides, cyclodextrins, fatty acids, pyrrolidines, azones, sulfoxides, terpenes and the like.

Suitable active carrier molecules may include one or more from the group comprising piperine and the like.

Suitable chelating or complexing agents may include one or more from the group comprising cyclodextrin, ethylenediamine tetra acetic acid or derivatives/salts thereof, e.g. disodium edetate, dihydroxyethyl glycine, glucamine, citric acid, tartaric acid, gluconic acid, phosphoric acid and the like.

The prime aspect of the present invention is that the pharmaceutical composition establishes a fast release pattern owing to kind of solubilizes and suspending or dispersing agents used in the formulation and thereby producing an immediate burst release that will in turn to increase in bioavailability at initial release.

One aspect of the present invention may include a pharmaceutical composition comprising about 0.1 mg to 500 mg of Nintedanib or pharmaceutically acceptable salt thereof with pharmaceutically acceptable excipients.

In yet another aspect of the present invention, wherein the pharmaceutical composition manufactured is homogenous dispersions, which results in to enhanced in-vitro dissolution profile.

Another aspect according to the present invention, wherein the formulated product manufactured is homogenous dispersions, which results in to enhanced in-vitro dissolution profile about 80% to 95% within initial 15 minutes of drug release, preferably more than 90% within initial 15 minutes of drug release.

In yet another aspect of the present invention, wherein the pharmaceutical composition manufactured by number of stages in manufacturing process including mixing and homogenization.

Additional aspect according to the present invention is that the formulated product is a stabilized homogenous oily dispersion.

In one aspect, process for preparation of an oral pharmaceutical composition as per the present invention comprising following steps:
a) Melting Lauroyl macrogol (or polyoxyl) 6 glycerides (Labrafil M 2130 CS);
b) mixing medium chain fatty acid triglycerides (Labrafac Lipofile WL 1349) along with Nintedanib esylate with melted Labrafil M 2130 CS obtained in step-1 and stirred continuously until it became homogeneous dispersion;
c) The homogenous dispersion prepared in step-2, was then encapsulated in soft-gelatin capsule.

In one aspect process for preparation of an oral pharmaceutical composition as per the present invention comprising preparation of soft-gelatin capsule wherein content of gelatin is not more than 50% of total ingredients.

The invention will be further described with respect to the following examples; however, the scope of the invention is not limited thereby. While the present invention has been described in terms of its specific embodiments, certain modifications and equivalents will be apparent to those skilled in the art and are intended to be included within the scope of the present invention.

### EXAMPLES

Following section now will describe various formulation example as well as test examples by which the formulation example were evaluated. Further, results of the present invention were compared with the marketed formulation as well as nearest prior-arts.

### Example-1

| | | Qty. per Capsule |
|---|---|---|
| Sr. No. | Ingredients | (mg) |
| 1. | Nintedanib esylate** | 180.60 |
| 2. | Medium chain fatty acid triglycerides | 239.40 |
| | (Labrafac Lipofile WL 1349) | |

| | |
|---|---|
| Total | 420.00 |

**180.60 mg of Nintedanib esylate is equivalent to 150 mg of Nintedanib free base.

### Process for Preparation:

1. Nintedanib esylate was added into medium chain fatty acid triglycerides (Labrafac Lipofile WL 1349) and stirred in magnetic stirrer for about 2 hours;
2. The homogeneous dispersion prepared in step-1 was then encapsulated in soft-gelatin capsule.

### Example-2

| | | Qty. per Capsule |
|---|---|---|
| Sr. No. | Ingredients | (mg) |
| 1. | Nintedanib esylate** | 180.60 |
| 2. | Medium chain fatty acid triglycerides | 162.90 |
| | (Labrafac Lipofile WL 1349) | |
| 3. | Hydrogenated vegetable oil | 76.50 |

| | |
|---|---|
| Total | 420.00 |

**180.60 mg of Nintedanib esylate is equivalent to 150 mg of Nintedanib free base.

### Process for Preparation:

1. Hydrogenated oil was added into medium chain fatty acid triglycerides (Labrafac Lipofile WL 1349) and stirred continuously until it became homogeneous solution;
2. Nintedanib esylate was added slowly to the homogenous solution prepared in step-1 under continuous stirring;
3. The stirring was continued until 3 hours to form homogeneous dispersion;
4. The homogeneous dispersion prepared in step-3, was then encapsulated in soft-gelatin capsule.

### Example-3

| | | Qty. per | |
|---|---|---|---|
| Sr. | | Capsule | |
| No. | Ingredients | (mg) | Function |
| 1. | Nintedanib esylate** | 180.60 | API |
| 2. | Medium chain fatty acid triglycerides | 162.90 | Vehicle |
| | (e.g. Labrafac Lipofile WL 1349) | | |
| 3. | Lauroyl polyoxyl-6-glycerides | 76.50 | Suspending or |
| | (e.g. Labrafil M 2130 CS) | | dispersing agent |

| | |
|---|---|
| Total | 420.00 |

**180.60 mg of Nintedanib esylate is equivalent to 150 mg of Nintedanib free base.

### Process for Preparation:

1. Lauroyl polyoxyl-6-glycerides (e.g. Labrafil M 2130 CS) was melted at the temperature around 45±5° C.;
2. Melted lauroyl polyoxyl-6-glycerides (e.g. Labrafil M 2130 CS) obtained in step-1, was mixed with medium chain fatty acid triglycerides (e.g. Labrafac Lipofile WL 1349) along with Nintedanib esylate and stirred continuously until it became homogeneous dispersion;
3. The homogeneous dispersion prepared in step-2, was then encapsulated in soft-gelatin capsule.

### Example-4

| | | Qty. per | |
|---|---|---|---|
| Sr. | | Capsule | |
| No. | Ingredients | (mg) | Function |
| 1. | Nintedanib esylate** | 120.40 | API |
| 2. | Medium chain fatty acid triglycerides | 108.60 | Vehicle |
| | (Labrafac Lipofile WL 1349) | | |
| 3. | Lauroyl polyoxyl-6-glycerides | 51.00 | Suspending or |
| | (Labrafil M 2130 CS) | | dispersing agent |

| | |
|---|---|
| Total | 280.00 |

**120.40 mg of Nintedanib esylate is equivalent to 100 mg of Nintedanib free base.

### Process for Preparation:

1. Lauroyl polyoxyl-6-glycerides (Labrafil M 2130 CS) was melted at the temperature around 45±5° C.;
2. Melted Lauroyl polyoxyl-6-glycerides (Labrafil M 2130 CS) obtained in step-1, was mixed with medium chain fatty acid triglycerides (Labrafac Lipofile WL 1349) along with Nintedanib esylate and stirred continuously until it became homogeneous dispersion;
3. The homogeneous dispersion prepared in step-2, was then encapsulated in soft-gelatin capsule.

### Example-5

| | | Qty. per | |
|---|---|---|---|
| Sr. | | Capsule | |
| No. | Ingredients | (mg) | Function |
| 1. | Nintedanib esylate** | 180.60 | API |
| 2. | Labrasol ® ALF | 162.90 | Vehicle |
| 3. | Lauroglycol ™ FCC | 76.50 | Suspending or |
| | | | dispersing agent |

| | |
|---|---|
| Total | 420.00 |

**180.60 mg of Nintedanib esylate is equivalent to 150 mg of Nintedanib free base.

### Process for Preparation:

1. Lauroglycol™ FCC was melted at the temperature around 45±5° C.;
2. Melted Lauroglycol™ FCC obtained in step-1, was mixed with Labrasol® ALF along with Nintedanib esylate and stirred continuously until it became homogeneous dispersion;
3. The homogeneous dispersion prepared in step-2, was then encapsulated in soft-gelatin capsule.

### Test Example-1: Dissolution Profile Comparison

Comparison of the dissolution profile study was incorporated to evaluate the superiority of the composition prepared as per the present invention against marketed formulation (OFEV® 150 mg) as well as example-3 and 4 of the only prior-art (WO2019/197961) which has reported dissolution data in the dissolution media (OGD media) recommended by USFDA.

For this comparative study, dissolution profile of the soft gelatin capsule prepared according to example-3 (150 mg), OFEV®-150 mg (RLD), example-3 of WO2019/197961 (150 mg) and example-4 of WO2019/197961 (150 mg) were observed in media as defined by USFDA, which is having 900 ml volume of 0.1 N HCl dissolution media in USP apparatus type II (paddle) at 100 RPM at 37° C. The obtained dissolution profile results are reported in below table.

Comparison of dissolution Profile for
Nintedanib esylate 150 mg formulations

| Dissolution Media | | 0.1N HCl | | |
|---|---|---|---|---|
| Dissolution Media Volume | | 900 ml | | |
| USP Apparatus | | Type-II (Paddle with sinker) | | |
| RPM | | 100 RPM | | |
| | % Drug release | | | |
| | | | WO2019/197961 | |
| Time | Example-3 of the | OFEV ® | Exam- | Exam- |
| (min) | present invention | (WO2009/147212) | ple-3 | ple-4 |
| 10 | 70 | 67 | 73 | 74 |
| 15 | 97 | 83 | 83 | 81 |
| 20 | 98 | 96 | 91 | 88 |
| 30 | 99 | 97 | 99 | 96 |
| 45 | 99 | 98 | 99 | 96 |
| 60 | 99 | 98 | 99 | 97 |

Formulation Perspective

| | | |
|---|---|---|
| Lecithin | Devoid of | Lecithin is essentially added in identical |
| Content | Lecithin | proportion in all three compositions |

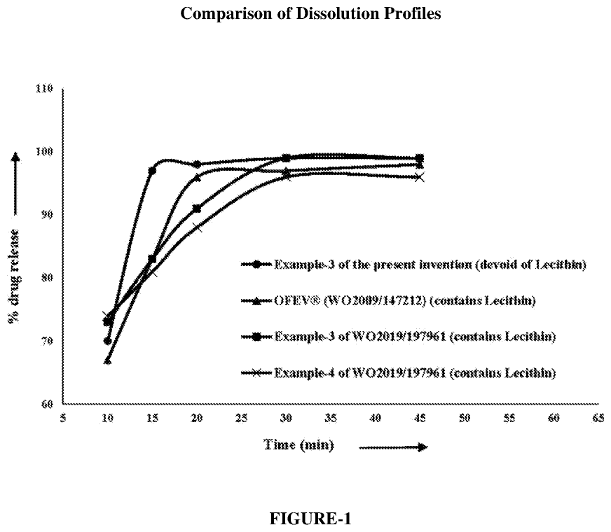

As per the above comparison, there is clear indication that the dissolution profile of the composition as per the present invention (which is devoid of Lecithin) is superior to all the other prior-arts (which contain lecithin as an essential part in identical proportion). Therefore, above results demonstrate that the present novel invention surprisingly produces better results and proves its inventiveness by technical advancement against the nearest prior-art publications. A Graphical representation of above dissolution profile comparative study is reported in FIG. 1.

### Test Example-2: Comparison of Assay in Accelerated Stability Study Conditions

The stability study of the composition as per the present invention, OFEV®-150 mg (RLD), example-3 of WO2019/197961 (150 mg) and example-4 of WO2019/197961 (150 mg) were observed. All the samples were charged for stability study at accelerated condition at ±2° C. and 75±5% RH and chemical parameters were evaluated at initial, after 1 month, after 6 months and after 9 months. The stability results obtained are reported in below table.

Comparison of assay for Nintedanib esylate 150 mg formulations
Accelerated stability conditions

| Temperature range | | 40 ± 2° C. | | |
|---|---|---|---|---|
| % Relative Humidity (% RH) | | 75 ± 5% RH | | |
| Time Interval | | Initial, after 1 month, after 6 months, | | |
| | | after 9 months | | |
| | % Assay | | | |
| | Example-3 of | | WO2019/197961 | |
| | the present | WO2009/147212 | Exam- | Exam- |
| Time Interval | invention | OFEV ® | ple-3 | ple-4 |

Assay

| | | | | |
|---|---|---|---|---|
| Initial | 99.51 | 99.60 | 99.20 | 99.10 |
| After 1 month | 99.42 | NP | 99.30 | 99.70 |
| After 6 months | 99.16 | NP | 100.7 | 98.50 |
| After 9 months | 99.05 | NP | 99.10 | 98.25 |

Formulation Perspective

| | | |
|---|---|---|
| Lecithin | Devoid of | Lecithin is essentially added in identical |
| Content | Lecithin | proportion in all three compositions |

Wherein, NP = Not Performed

As per the above comparison, there is clear indication that the % assay of the composition as per the present invention (which is devoid of Lecithin) is showing superior results to all the other prior-arts (which contain lecithin as an essential part in identical proportion) even after 9 months of accelerated stability conditions. Therefore, above results demonstrate that the present novel invention surprisingly produces better results and proves its inventiveness by technical advancement against the nearest prior-art publications.

### Test Example-3: Comparison of Impurity Profile in Accelerated Stability Study Conditions

The impurity profile of the composition as per the present invention, OFEV®-150 mg (RLD), example-3 of WO2019/197961 (150 mg) and example-4 of WO2019/197961 (150 mg) were observed. All the samples were charged for stability study at accelerated condition at 40±2° C. and 75±5% RH and chemical parameters were evaluated at initial, after 1 month, after 6 months and after 9 months. The impurity profile obtained are reported in below table.

Comparison of impurity profile for Nintedanib esylate 150 mg formulations
Accelerated stability conditions

| Temperature range | 40 ± 2° C. | | | |
|---|---|---|---|---|
| % Relative Humidity (% RH) | 75 ± 5% RH | | | |
| Time Interval | Initial, after 1 month, after 6 months, after 9 months | | | |
| | Impurity Profile | | | |
| | Example-3 of | WO2009/ | WO2019/ | |
| Time | the present | 147212 | 197961 | |
| Interval | invention | OFEV ® | Example-3 | Example-4 |

Impurity Profile

| Impurity | | | | | | | | |
|---|---|---|---|---|---|---|---|---|
| type | Acid | Total | Acid | Total | Acid | Total | Acid | Total |
| Initial | 0.003 | 0.108 | ND | 0.113 | 0.055 | 0.186 | 0.053 | 0.179 |
| After 1 | 0.003 | 0.158 | ND | 0.089 | 0.051 | 0.204 | 0.053 | 0.149 |
| month | | | | | | | | |
| After 6 | 0.004 | 0.108 | ND | 0.093 | 0.059 | 0.187 | 0.058 | 0.172 |
| months | | | | | | | | |
| After 9 | 0.005 | 0.133 | ND | 0.099 | 0.062 | 0.194 | 0.063 | 0.191 |
| months | | | | | | | | |

Formulation Perspective

| | | |
|---|---|---|
| Lecithin | Devoid of | Lecithin is essentially added in identical |
| Content | Lecithin | proportion in all three compositions |

Wherein,
ND = Not Detected

As per the above comparison, there is clear indication that the impurity profile of the composition as per the present invention (which is devoid of Lecithin) is showing superior results to all the other prior-arts (which contain lecithin as an essential part in identical proportion) even after 9 months of accelerated stability conditions. Therefore, above results demonstrate that the present novel invention surprisingly produces better results once again and proves its inventiveness by technical advancement against the nearest prior-art publications.

The invention described herein comprises in various objects as mentioned above and their description in relation to characteristics, compositions and process adopted. While these aspects are emphasised in the invention, any variations of the invention described above are not to be regarded as departure from the spirit and scope of the invention as described.

The above-mentioned examples are provided for illustrative purpose only and these examples are in no way limitative on the present invention.

1. An oral pharmaceutical formulation comprising of oily dispersion which consisting essentially of:
a) Nintedanib or pharmaceutically acceptable salt thereof;
b) Medium chain fatty acid glycerides;
wherein the above formulation,
i) does not contain Lecithin;
ii) shows more than 90% of drug dissolution within 15 minutes; and,
iii) shows total impurities content less than 0.2% after stability study of 9 months at (40±2)° C. and (75±5)% RH.

2. The oral pharmaceutical formulation comprising of oily dispersion of Nintedanib or pharmaceutically acceptable salt thereof as claimed in claim 1, wherein the ratio of Nintedanib esylate to medium chain fatty acid glycerides is not more than 1:1.

3. The oral pharmaceutical formulation comprising of oily dispersion of Nintedanib or pharmaceutically acceptable salt thereof as claimed in claim 1, wherein the formulation further comprises Lauroyl polyoxyl-6-glycerides.

4. The oral pharmaceutical formulation comprising of oily dispersion of Nintedanib or pharmaceutically acceptable salt thereof as claimed in claim 3, wherein the ratio of Nintedanib esylate to Lauroyl polyoxyl-6-glycerides is not more than 1:0.5.

5. The oral pharmaceutical formulation comprising of oily dispersion of Nintedanib or pharmaceutically acceptable salt thereof as claimed in claim 1, wherein the formulation shows total impurities less than 0.2% after stability study of 9 months at (40±2)° C. and (75±5)% RH in 900 ml volume of 0.1 N HCl dissolution media in USP apparatus type II (paddle) at 100 RPM at 37° C.

6. The oral pharmaceutical formulation comprising of oily dispersion of Nintedanib or pharmaceutically acceptable salt thereof as claimed in claim 1, wherein the formulation shows % assay not less than 99% after the stability study of 9 months at (40±2)° C. and (75±5)% RH.

7. The oral pharmaceutical formulation comprising of oily dispersion of Nintedanib or pharmaceutically acceptable salt thereof as claimed in claim 1, wherein Nintedanib or pharmaceutically acceptable salt thereof is in crystalline form or in amorphous form.

8. An oral pharmaceutical formulation comprising of oily dispersion of Nintedanib Esylate having following formula:

| Ingredients | Qty. per Capsule (% w/w) |
|---|---|
| Nintedanib esylate | 43.00 |
| Medium chain fatty acid triglycerides | 38.79 |
| Lauroyl polyoxyl-6-glycerides | 18.21 |

9. An oral pharmaceutical formulation comprising of oily dispersion of Nintedanib Esylate having following formula:

| Ingredients | Qty. per Capsule (mg) |
|---|---|
| Nintedanib esylate | 180.60 |
| Labrafac Lipofile WL 1349 | 162.90 |
| Labrafil M 2130 CS | 76.50 |

10. The oral pharmaceutical formulation comprising of oily dispersion of Nintedanib Esylate as claimed in claim 9, wherein the process for the preparation of the said formulation is prepared by following steps:
a) melting of Labrafil M 2130 CS at the temperature around 45±5° C.;
b) mixing of Lbrafac Lipofile WL1349 along with above melted solution and Nintedanib Esylate to form homogeneous dispersion;
c) encapsulating above homogeneous dispersion to provide the final formulation.
